(19) United States
(12) Patent Application Publication
(10) Pub. No.: US 2007/0078391 A1
(43) Pub. Date: Apr. 5, 2007

(54) Implantable medical device

(75) Inventors: Ronald Wortley, Salt Lake City, UT (US); Eric King, West Jordan, UT (US); Giorgio di Palma, Queensbury, NY (US)

Correspondence Address: BOND, SCHOENECK & KING, PLLC, ONE LINCOLN CENTER, SYRACUSE, NY, 13202-1355, US

(73) Assignee: AngioDynamics Inc., Queensbury, NY (US)

(21) Appl. No.: 11/536,363

(22) Filed: Sep. 28, 2006

Related U.S. Application Data
(60) Provisional application No. 60/722,800, filed on Sep. 30, 2005.

(51) Int. Cl. A61M 5/00

(52) U.S. Cl. 604/116; 604/288.02

(57) ABSTRACT

A medical device suitable for subcutaneous implantation, generally including a housing having a reservoir, a septum positioned within and supported by the housing, at least one light emitting element placed in position defining relation to the septum, and a pressure actuated, light activating circuitry associated with the at least one light emitting element. The light element(s) may be positioned, for instance, in at least partially surrounding relation around the septum, embedded within or below a translucent housing that supports the septum, positioned within the reservoir and adapted to emit its light through a translucent septum, or positioned on the exterior of the supporting housing. The medical device can be adapted to receive high pressure fluid injections and if so adapted, will include a light emitting element that will provide a visual indication this capacity.

### PRIORITY CLAIM

The present application claims priority to U.S. Provisional Application Ser. No. 60/722,800, filed Sep. 30, 2005, the entirety of which is hereby incorporated by reference.

### BACKGROUND OF THE INVENTION

1. Field of Invention

The present invention relates generally to implantable medical devices, and more particularly to vascular access devices, such as ports, and methods associated with such devices.

2. Description of the Art

As known in the art, vascular access systems are used to provide recurring access to the body of a patient when performing various therapeutic or diagnostic procedures. The vascular access system typically contains a vascular access port and an elongated, pliable catheter that is coupled to the port. The port is implanted in shallow tissue areas in the body of patient, such as subcutaneously under the skin. The entire device is located subcutaneously to enhance a patient's quality of life. Because the vascular access port is implanted subcutaneously, it cannot be seen outside the body.

The port catheter is inserted into the vascular system at the desired location in the patient and is used to infuse a desired substance to the necessary location in the body of the patient. A needle and a hypodermic syringe or other fluid source is used to deliver medication through the skin and soft tissue to a fluid reservoir in the vascular access port. The medication flows through the catheter and is discharged within the body at the distal end of the catheter.

Alternatively, the vascular access system can be used to withdraw body fluids by a reverse process. A typical vascular access port has a housing, a septum through which a needle is inserted, and a base containing a fluid reservoir, as is well understood in the art.

Because vascular access systems are implanted in the tissue of a patient for long periods of time, they are typically made as small as possible. A small profile port reduces patient discomfort, thus making any medical procedure using them as minimally invasive as possible. Shrinking the size of these systems also requires shrinking the size of the vascular access port injection site or “needle target”. As ports become smaller and smaller, or are located deeper in the tissue, it becomes more and more difficult to locate the proper insertion site or “needle target” required to infuse the desired medications through the tissue into the port reservoir. This often results in unnecessary and repetitive insertions of the needle into the patient before the correct site is located allowing the needle to enter the port reservoir. It is also difficult for the health care provider to know when the correct site has been accessed, as this is not an image-guided procedure.

In addition to above described uses of ports, these implantable medical devices may also be used as a conduit for contrast media used in Computer Tomography (CT) imaging processes. CT is a common medical imaging modality for diagnostic assessments that produces cross-sectional images or slices using X-ray technology. CT without contrast media allows imaging of bones (similar to X-ray), but will not provide adequate imaging of soft tissue structures, such as tumors, organs and vasculature. Thus, CT imaging may be enhanced by using an injection of contrast media into the body to improve visibility of soft tissue structures. Typically, contrast media is injected into the patient through a needle inserted in a peripheral vein. PICC lines or vascular access ports can also be used but these devices must be able to withstand the high pressures required for CT injections.

Contrast-enhanced CT requires high pressure, high flow rate contrast injections rates to ensure sufficient tissue uptake of the contrast agent, necessary to achieve adequate visibility of the tissue structures. Using a CT injector, a large volume of contrast media is injected under high pressures into the vascular access port. A typical CT injector may produce injection pressures of between 300-350 psi at the pump outlet.

A standard vascular access port can withstand only about 25 psi. If the injection pressure exceeds the tolerance of the septum, the septum may rupture, the catheter may fail, or the catheter tip may become displaced. Ruptures may lead to serious complications or injuries to the patient, including leaking or extravasation of the contrast media into the port pocket and surrounding tissue, resulting in clinically significant complications, caused by tissue necrosis from exposure to contrast media. Ruptures can also result in the loss of venous access requiring vascular access device replacement and potential complications from a second interventional procedure.

Vascular access ports have recently been designed to withstand the higher pressures generated by CT injections. Although these ports have successfully addressed the issues of maintaining septum and overall port integrity after repeated high-pressure injections, prior art port designs have not addressed the problem that medical practitioners have with being able to accurately identify an implantable port as CT-injectable. Unlike high-pressure PICC lines in which the external segment of the catheter can easily be labeled by the manufacturer as either a standard line or a high-pressure injectable line, a vascular access port is completely implanted within the patient and cannot be visibly labeled as CT-injectable. Accordingly, there is a need to provide a vascular port with a readily visible CT-identification feature to allow the practitioner to easily determine if high-pressure injections can be administered through the port septum.

3. Objects and Advantages

It is therefore a principal object and advantage of the present invention to provide an implantable medical device for vascular access that contains a system for non-invasively guiding treatment personnel to the access location.

It is a further object and advantage of the present invention to provide a non-invasive guidance system that can be incorporated into existing designs of the same sort of medical devices.

It is an additional object and advantage of the present invention to provide an implantable medical device for vascular access that may be used for high pressure fluid injections, and is distinctly identifiable as such.

It is another object and advantage of the present invention to provide a vascular access medical device that verifies proper access of the device.

It is another object and advantage of the present invention to provide a vascular access medical device that provides a visual indication of device malfunction.

Other objects and advantages of the present invention will in part be obvious and in part appear hereinafter.

### SUMMARY OF THE INVENTION

In accordance with the foregoing objects and advantages, one aspect of the present invention provides a medical device suitable for subcutaneous implantation, such as a vascular access port, generally comprising a housing, a septum positioned within and supported by the housing, at least one light emitting element positioned in position-defining relation to the septum, and pressure actuated, light activating circuitry associated with the at least one light emitting element. The light emitting element(s) may be positioned, for instance, in at least partially surrounding relation around the septum or in aligned relation with the septum so long as when light is emitted therefrom, it is possible for the light to be observed and the position of the septum to be determined based upon that observance of light. The pressure to actuate the circuitry can be applied by medical personnel applying pressure to the device, but would preferably be actuated either by vertical compression or by pressure applied to the sides of the housing for ease of operation.

In one embodiment of the invention the light activating circuitry generally comprises a light supporting member that extends in a first plane and includes a conductive pathway formed thereon, and a first plate extending in a first plane transverse and in connected relation to the light supporting member. In this aspect of the invention, a second plate connected to the light supporting member and extending in a plane parallel to and laterally spaced from the first plate may also be included. Conductive traces formed on the first and second plates together with a conductive pathway formed on the light supporting member which is contiguous with the conductive traces form a circuit that may be selectively closed by application of pressure to the device, thereby actuating the light emitting elements that are securely positioned on the conductive pathway formed on the light supporting member.

In another embodiment of the invention, the light actuating circuitry generally comprises a first portion that extends in a first plane and that includes a conductive pathway formed thereon, a second portion that extends in a second plane parallel to said first plane; and a third portion that extends between and interconnects said first portion and said second portion. In this aspect of the invention, a power source is operably positioned on the second portion, and a circuit comprising the conductive pathways that are contiguous through the first, second and third portions is selectively closed by compressing the device along its vertical axis.

In another embodiment, the light actuating circuitry generally comprises light activating circuitry comprises a first portion that extends about the septum and includes a conductive pathway formed thereon, and a second portion connected to the first portion and that includes positive and negative terminals mounted thereon. The first portion forms a partial ring/track around the septum and includes first and second pressure switches on opposing sides thereof. The second portion contains a conductive pathway that provides a means to transport power from a power source to the first and second switches. Upon application of pressure to the sides of the device the circuit that carries power from the power source to the first and second switches is closed, thereby actuating the at least one light emitting element.

In another aspect of the present invention a medical device suitable for a predetermined use and for subcutaneous implantation is provided, such as a vascular access port that has the capacity to withstand a high pressure fluid injection. In this aspect of the invention, the medical device generally comprises a housing, a septum positioned within and supported by said housing, and having the capacity to be used for the predetermined use, at least one first light emitting element associated with said housing and adapted to identify the capacity of the medical device to be used for the predetermined use, and light activating circuitry. In this aspect of the invention, the at least one light emitting element is adapted to exhibit a predetermined characteristic, such as emitting light of a distinct color, that will provide a visible indication to medical personnel who can observe the light through the patient's skin that the device is or is not suitable for receiving a high pressure fluid injection.

Another aspect of the invention includes a method for non-invasively determining the location of a medical device implanted subcutaneously in a patient, wherein the medical device comprises a housing, a septum positioned within and supported by the housing, at least one light emitting element, and light activating circuitry associated with the at least one light emitting element, with the method comprising the step of applying pressure to the medical device that results in actuation of the at least one light emitting element. Following actuation of the light emitting elements, the location of the septum is determined by visually observing the position of the at least one light emitting element. In furtherance of the aspect of the invention that provides a visual cue that the device can be used for a predetermined purpose, such as whether it can withstand a high pressure fluid injection, after actuation of the light emitting element, the method includes determining whether the device can be used for the predetermined purpose based upon visual observation of a second light emitting element. The light emitting elements may remain on for a predetermined period of time following release of the pressure that actuated the circuit (or the lights can also be deactivated at that time), but regardless, a needle may then be assuredly passed through the septum.

In a further aspect of the present invention, a method for determining whether a medical device that is adapted for subcutaneous implantation in a patient has been impaired is provided. The medical device generally comprises a housing, a septum positioned within and supported by the housing, at least one light emitting device, and light activating circuitry operably coupled to the at least one light emitting element. The method of determining whether the device has been impaired generally comprises the steps of incorporating a predetermined sensor in the housing that is adapted to quantitatively measure a predetermined physical condition and compare the quantitative measurement to a predetermined threshold, and actuating the light activating circuitry in the event the predetermined threshold has been exceeded, thereby causing the at least one light emitting element to emit light. The sensors can be, for example, pressure based sensors (i.e., pressure transducers), or impedance-based sensors capable of measuring the impedance in the interior of and the exterior to the medical device.

### BRIEF DESCRIPTION OF THE DRAWINGS

The present invention will be more fully understood and appreciated by reading the following Detailed Description in conjunction with the accompanying drawings, in which:

FIG. 1 is a perspective view of a vascular access port in accordance with one embodiment of the present invention;

FIG. 2 is a top plan view of the vascular access port of FIG. 1.

FIG. 3 is a side elevation view of the vascular access port of FIG. 1;

FIG. 4 is a cross-sectional view taken along lines 4-4 of FIG. 2;

FIG. 5 is an exploded perspective view of the vascular access port of FIG. 1.

FIG. 6A is a perspective view of the LED circuit prior to assembly;

FIG. 6B is a perspective view of the LED circuit in its assembled form;

FIGS. 7A and 7B are schematic representations of the LED circuit;

FIG. 8 is a perspective view of a second embodiment of the present invention;

FIG. 9 is a top plan view of a second embodiment of the present invention;

FIG. 10 is a side elevation view thereof;

FIG. 11 is a cross-sectional view taken along line 11-11 of FIG. 9;

FIG. 12 is an exploded perspective view of the second embodiment;

FIG. 13A is a perspective view of the LED circuit of the second embodiment in its assembled form;

FIG. 13B is a perspective view of an LED circuit with battery and pressure plate;

FIG. 14 is a perspective view of a third embodiment of the present invention;

FIG. 15 is a top plan view thereof;

FIG. 16 is a side elevation view thereof;

FIG. 17 is a cross-sectional view taken along line 17-17 of FIG. 15;

FIG. 18A is a cross-sectional view taken along lines 18-18 of FIG. 15;

FIG. 18B is an enlarged cross-sectional view of the encircled portion of FIG. 18A, without the lower housing and battery;

FIG. 19 is an exploded perspective view thereof;

FIG. 20 is a perspective view of the LED circuit of the third embodiment in its assembled form;

FIGS. 21-23 are perspective views illustrating the method of using the present invention; and

FIG. 24 is a high level flow chart illustrating the method of using the present invention.

### DETAILED DESCRIPTION

The following description provides specific details in order to provide a thorough understanding of the invention. The skilled artisan, however, would understand that the invention can be practiced without employing these specific details. Indeed, the invention can be practiced by modifying the illustrated methods and resulting products and can be used in conjunction with apparatuses and techniques conventionally used in the industry. The invention, however, could easily be adapted for any subcutaneous medical device which requires visual confirmation of the location.

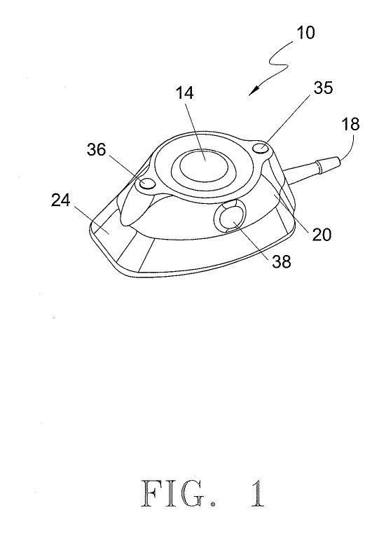

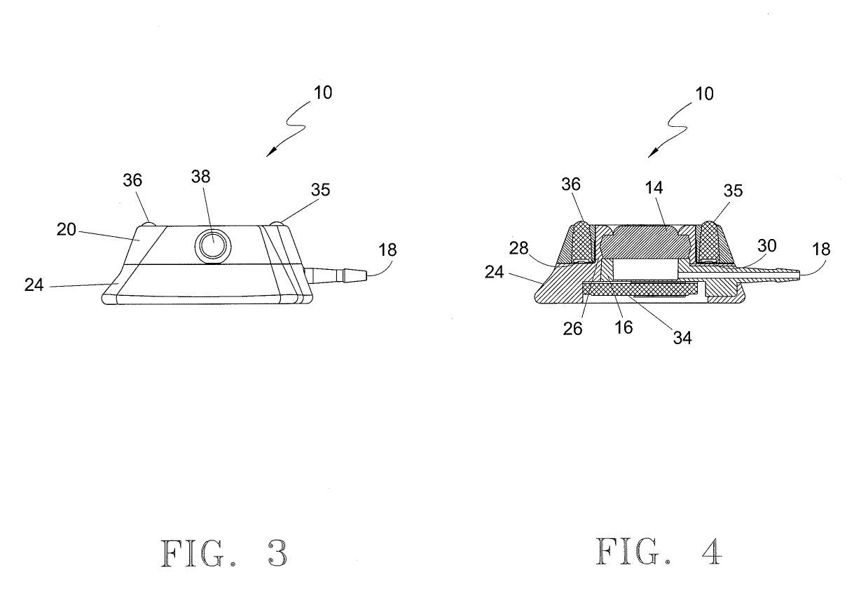

Referring now to the drawings, in which like reference numerals refer to like parts throughout, there is seen in FIGS. 1-7 an implantable vascular access port, designated generally by reference numeral 10, essentially comprising an upper housing 20 and a lower housing 24. Lower housing 24 supports and surrounds a septum 14 that is, in turn, positioned in vertically aligned relation above a port can 16. As is conventional with vascular access ports, septum 14 provides a needle injection and stabilizing point for fluid to be introduced into and removed from the venous system. After a needle passes through septum 14, fluid can be released therefrom by medical treatment personnel where it is contained by port can 16. An exit lumen 18 extends outwardly from port can 16 and delivers the fluid to the venous system through a catheter (not shown) that is fluidly connected to exit lumen 18.

In the first embodiment, and in fact in each of the embodiments of the present invention, port 10 includes a guidance system that non-invasively defines the location of septum 14 for purposes of providing a well-defined target for the medical personnel who need to insert a needle through the septum. In each of the embodiments the guidance system comprises at least one light emitting element that is incorporated into port 10 and is adapted to be viewable through the skin of the patient in which the port is subcutaneously implanted. By viewing the light that defines the septum location, the medical personnel will be able to accurately insert the needle through the septum without “trial and error.” The light can also be used to identify the port type, such as a CT-injectable port.

With reference to FIGS. 1-8, a first embodiment of port 10 is illustrated. In this embodiment, shown in an assembled state in FIGS. 1-5, port 10 comprises an upper housing 20 having a opening 22 formed centrally therethrough which surrounds lower housing 24, and lower housing 24 that is concentrically positioned within upper housing 20 and includes a cavity 25 that is defined by an upstanding side-wall 26 and in which port can 16 is positioned. Septum 14 is compressed between lower housing 24 and can 16. In addition to those elements common to ports and described hereinabove, port 10 further comprises a pair of light emitting diodes (LEDs) 28, 30, a conductive circuit 32 that interconnects LEDs 28, 30 to a power source 34 also incorporated into port 10 that provides the power for LEDs 28, 30.

More specifically, upper housing 20 includes a pair of diametrically opposed light guides 35, 36 to house and support LEDs 28, 30 that are positioned on opposite sides of opening 22, and therefore opposite sides of where septum 14 is positioned when port 10 is assembled. In addition, a pair of resilient, domed buttons 38, 40 are positioned on each side of upper housing 20 and serve as the manual actuation points for closing circuit 32. By manually depressing buttons 38 and 40 through the patient's tissue, the switches (as described below) on circuit 32 which are normally in an open position (thus not providing power to LEDs 28, 30) are closed, thereby providing power to LEDs 28, 30. When buttons 38 and 40 are released, they bias back to their neutral positions which once again opens the switches on circuit 32 cutting power to LEDs 28 and 30.

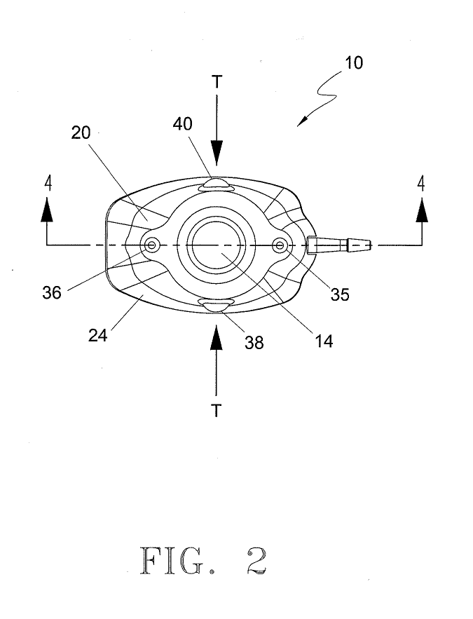

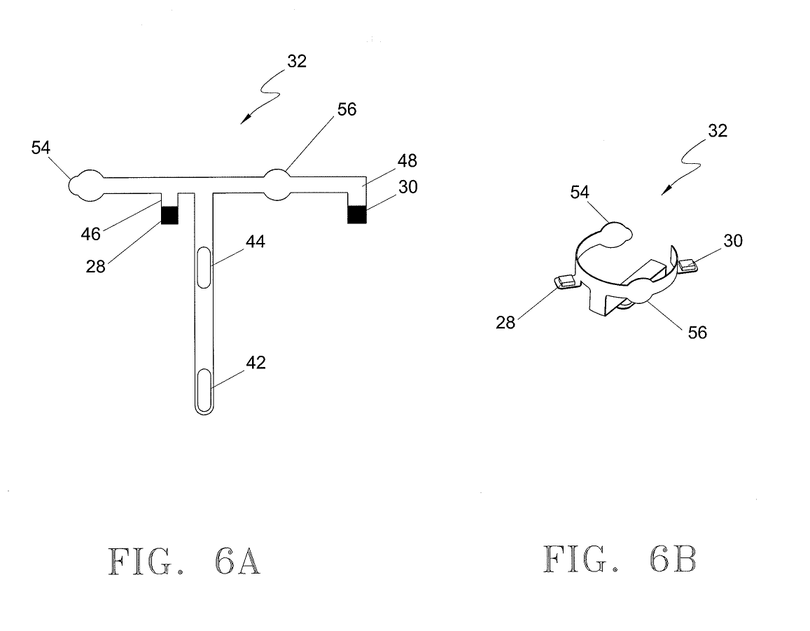

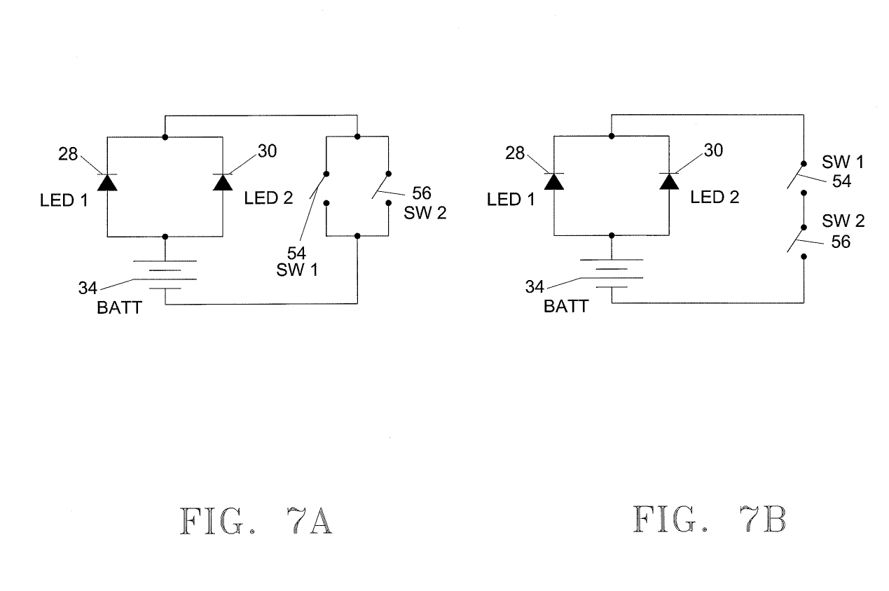

With reference to FIGS. 6A-B, LED circuit 32 is formed from a flexible, die-cut (essentially T-shaped) strip of material with conductive printing etched thereon to form the circuit, as shown schematically in FIGS. 7A and 7B (either a parallel or series arrangement can be used). The circuit 32 includes positive and negative contacts 42, 44 that are positioned in contacting relation to the positive and negative terminals of power source 34 when port 10 is assembled. A pair of LEDs 28, 30 are mounted to circuit 32 as shown in FIG. 6B. When port 10 is assembled, the two pressure switches 54, 56 on circuit 32 are positioned in radially inward spaced relation to buttons 38, 40, respectively. When buttons 38 and 40 are manually depressed (e.g., by a force applied along vector T shown in FIG. 2 that is transverse to the port's longitudinal axis), their inner surfaces contact switches 54, 56 which closes circuit 32, thereby providing power to LEDs 28, 30, respectively.

FIG. 7A illustrates a typical schematic of a circuit with parallel switching and FIG. 7B illustrates a schematic of a circuit with a series switch design. A parallel circuit requires only one of the two switches 54, 56 to be closed to activate the circuit and transmit power to the LEDs. By depressing a single button 38 or 40, the LEDs are activated. This design is advantageous in that it is easier for the medical practitioner to activate. As shown in FIG. 7B, a series circuit, on the other hand, requires closure of both switch 54 and switch 56 in order to activate the circuit and transmit power to the LEDs. The advantage of a series configuration is that since both buttons 38 and 40 must be depressed to activate the circuit, any inadvertent pressure applied to one button, such as might be caused by normal body movement, will not cause the LEDs to emit light. Either switch configuration is within the scope of this invention.

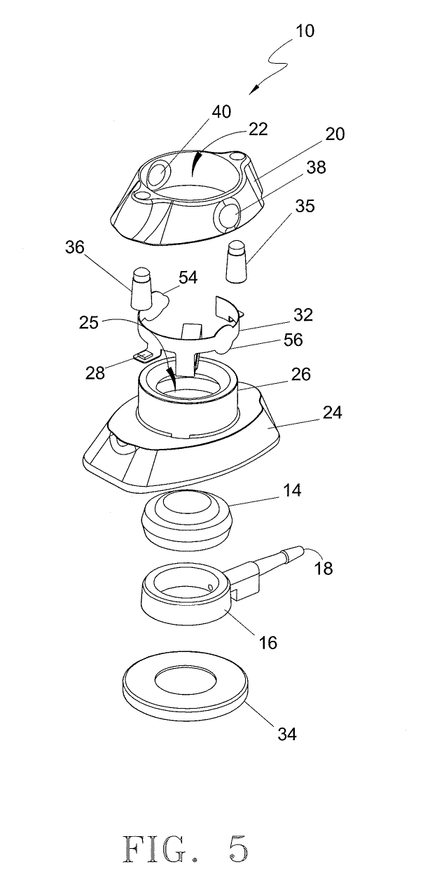

The assembly of port 10 is illustrated with reference to FIG. 5 and will further aid in understanding the structure of port 10. The first step in the assembly process is to assemble the lower housing 24 by inserting septum 14 and port can 16 through cavity 25 and securing them in place by, for example, welding. Septum 14 becomes compressed and sealed into position between can 16 and lower housing 24. Lower housing 24 includes a recess formed in the lower portion thereof through which exit lumen 18 extends. After securing the structural relationship between port can 16 and lower housing 24, the next step is to insert circuit 32 through lower housing 24 with negative and positive contacts 42, 44, (depicted in FIGS. 6A and 6B), positioned beneath housing 24 and nodes 46, 48 and switches 54, 56, being positioned above housing 24. Pressure switches 54, 56 and nodes 46, 48 can then be wrapped around side-wall 26 and circuit 32 can be adhesively secured to lower housing 24. The LEDs 28, 30 are permanently mounted to circuit 32 using a conductive adhesive or soldering technique commonly known in the art. Upper housing 20 can then be concentrically placed on top of lower housing 24 and bonded in place using a solvent. When upper housing 20 is joined with lower housing 24, pressure switches 54, 56 are positioned radially inward of upper housing 20 and radially outward of side-wall 26. The final two steps in the assembly process are to place power source (battery) 34 in the bottom of lower housing 24 and then bend the portion of circuit 32 containing the positive and negative terminals 42, 44 into contacting relation with the respective terminals on power source 34. Finally the power source assembly is encapsulated into the bottom of lower housing 24.

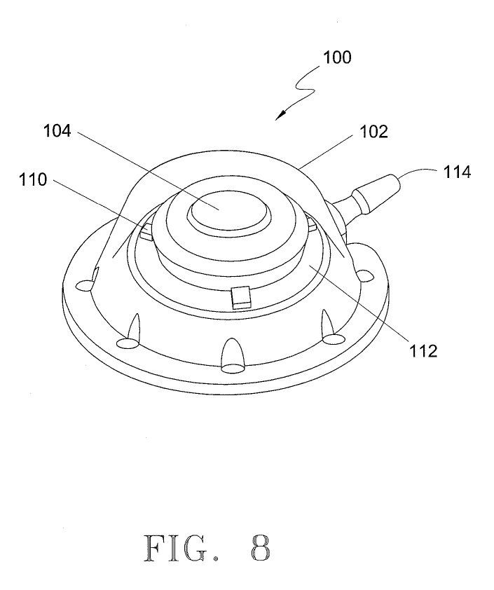

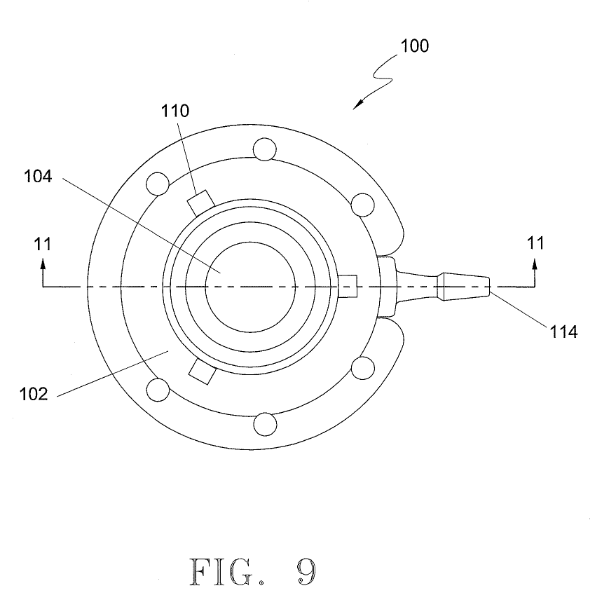

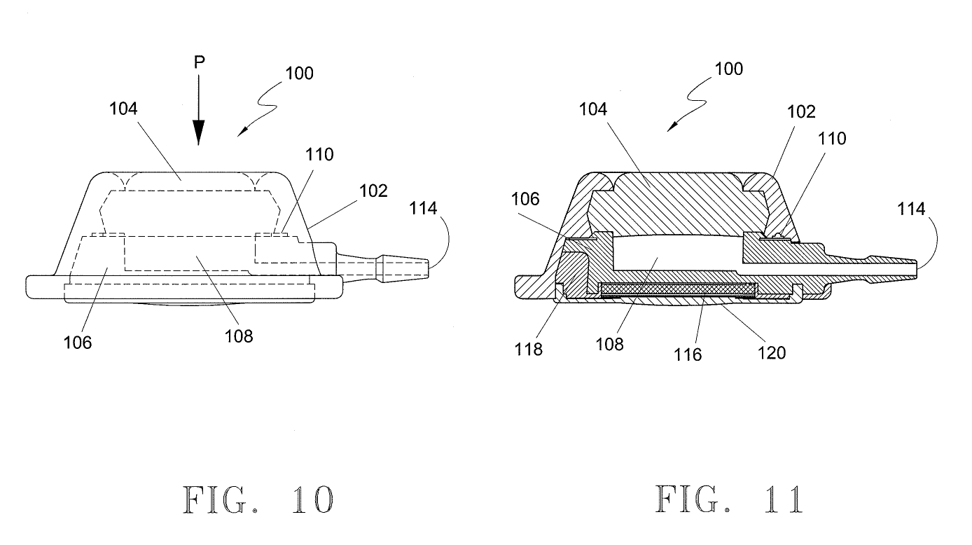

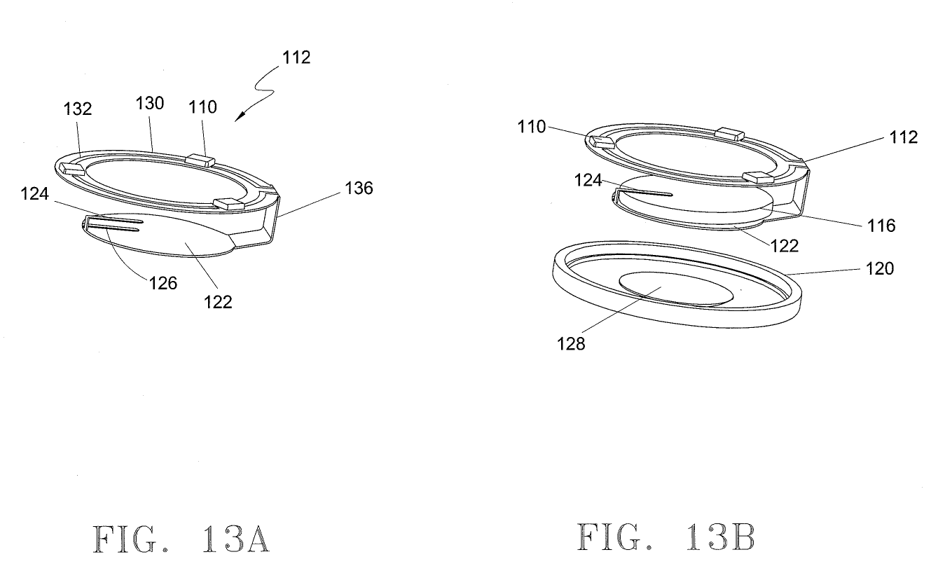

In another embodiment of the invention, the access port used in the systems of the invention is depicted in FIG. 8-13. In this embodiment, the light emitting elements are activated by applying pressure to the tissue located over the top of the port rather than applying pressure to side buttons. As depicted in FIG. 8-11, the access port 100 contains a housing 102 that supports a septum 104, and a base (or port can) 106 containing a fluid reservoir 108, which is connected to an exit lumen 114. These components are similar to those conventionally known and previously disclosed, and so, of course, can be used or adapted from components conventionally used and can be made from any materials conventionally used in such components.

Access port 100 also contains a lighting means that emits light from the access port. Any means that can emit light from the access port 100 can be used in this invention. In one aspect of the invention, the lighting means comprises light source 110 located on the upper surface of port can 106. The housing 102 may be of translucent or semi-translucent material to enhance visibility of the light source 110 when activated.

FIG. 12 illustrates an exploded view of the access port 100 comprising housing 102 surrounding and supporting port septum 104, a light source circuit 112, port can 106 positioned beneath septum 104, an exit lumen 114 extending outwardly from reservoir 108, a power source 116, a lower housing 118 and a pressure plate 120.

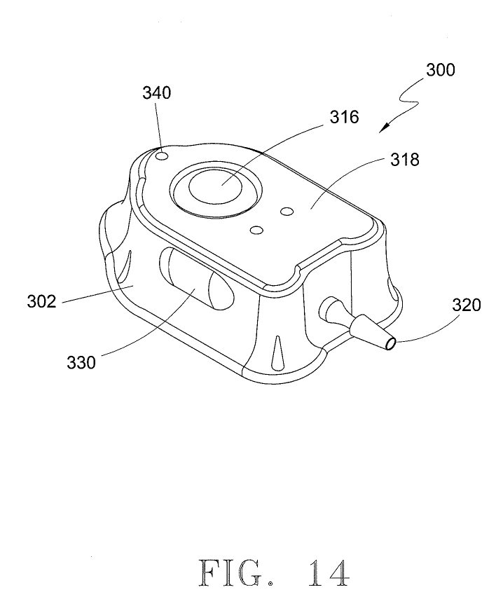

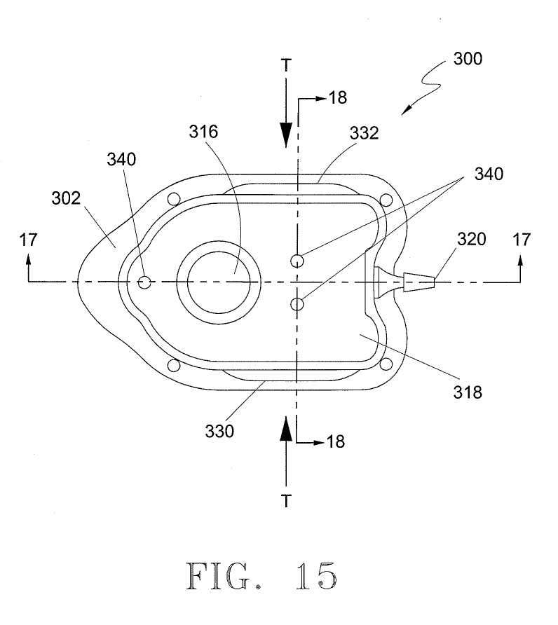

Depicted in FIG. 13A is a detail of light source circuit 112 showing the main components comprising the circuit 112, including the light emitting components 110, on/off conductive pad/pressure switch 122, and positive and negative terminals 124 and 126, respectively. Circuitry 112 electrically connects the light emitting components 110 to pressure switch 122. The pressure switch 122 controls contact between the positive and negative terminals 124 and 126, respectively, when the conductive pad/switch 122 is activated. Any light source containing at least one light element 110 can be used as the lighting means in the access port 100 (one element, for instance, could be implemented as a fiber optic strand that is positioned about the periphery of the septum).

FIG. 13B exhibits further details of the functionality of light source circuit 112 as an exploded view thereof. Power source 116 is positioned between positive and negative terminals 124 and 126, respectively, and is positioned above conductive pad/pressure switch 122. Pressure plate 120 contains a raised section 128 which activates conductive pad/pressure switch 122. Once access port 100 is subcutaneously implanted into the tissue, the health care provider can activate light source 110 by applying pressure on or around the implanted access port 100. The reaction force of the tissue under or around access port 100 is transferred through the raised section 128 of pressure plate 120 to the pressure switch 122. This action completes the circuit causing light sources 110 to illuminate, thereby making the injection site or “needle target” of septum 104 visible through the tissue. Once the health care provider releases pressure, the reaction force from the underlying tissue returns to zero. With no reaction force on the raised areas of pressure plate 120, conductive pad/pressure switch 122 opens causing the light elements' 110 illumination to cease. It should be noted that the pressure plate 120 could be located anywhere on the surface of access port 100, such as on each side of port housing 102. In this aspect of the design, the light component 110 would illuminate when force was transferred through the tissue to the sides of the port housing 102 during the palpating procedure to find the general port location.

Lighting components 110 can be any source of light known in the art. Examples of light components that can be used include incandescent bulbs, luminescent or fluorescent materials, and light emitting diodes (LEDs). In one aspect of the invention, LEDs are used for the light component 110.

The lighting means preferably contain more than a single light component. While theoretically any number of light components can be used, the number of light components 110 is selected so that the desired amount of light is obtained given the physical dimensions of access port 100. For example, when septum 104 with a diameter of about 1 centimeter is used, the number of light components can effectively be from 1 to 10. In one preferred aspect, the two to three light components 110 were found effective.

Light components 110 are arranged so that a desired amount, and theoretically the maximum of amount of light is emitted from access port 100. Thus, the orientation of light components 110 will depend on several factors, including the number of lights used, the desired direction of light emission, the materials used in access port 100 (through which the light may need to be transmitted), housing 102, and septum 104. In one aspect of the invention, light components 110 are arranged to create a substantially circular shape around the periphery of septum 104.

Light components 110 can be mounted at any location on access port 100 that provides the desired intensity of light, whether that is bright or dim. To obtain effective light transmittance, light components 110 are located on the outer, “upper” surface of housing 102. In another aspect of the invention, the light components are located between the port can 106 and housing 102 which is manufactured from a transparent/translucent thermal plastic, which permits light components 110 and accompanying circuit 112 to be encapsulated inside housing 102 while allowing light to transmit there through and into the surrounding tissue. This arrangement allows all the electronic components of port 100 to be safely contained within the device, thereby reducing or eliminating contact of these components with tissue.

Alternatively, the light components may be placed within the port reservoir, either on the bottom or on the inner surface of the vertical side-walls. In this embodiment, the light components emit visible light through the septum, illuminating the septum itself rather than the periphery of the septum.

The light emitted from port 100 can be any desired color or combination of colors. In one aspect of the invention, the presence of light-emitting elements may be used to identify the vascular access port as a device that meets the requirements for high-pressure fluid injections, such as used in CT. In another aspect of the invention, different colors are used to signify different parts of access port 100. For example, a second color (i.e., green) could be used in addition to a first color (i.e., red) that is used to locate the injection site. The additional, second color would be located at or above exit lumen 114 to indicate the location of the outlet relative to the injection site. This configuration would allow a health care provider to angle the needle towards exit lumen 114 if desired for more effective placement of medication, and also aid in inserting a wire to clear any blockages that may be in exit lumen 114. It should be noted that different colors penetrate tissue to different depths. A red color is typically the most visible under tissue, but other colors may be used depending on skin depth, color and personal preference.

In another aspect of the invention, different colors could be used to demonstrate different port sizes, configurations including multiple injection sites (e.g., at least two septa incorporated into the port), port types (e.g., a port capable of withstanding high pressure fluid injections such as is needed for CT), port materials, or specific types of indicated medicines. For instance, a particular color, red for instance, could be used to designate the port as being one that is designed to withstand injection of contrast media used in CT imaging.

The power source 116 can be any known in the art that provides the needed amount of power, yet will meet the size limitations needed for access port 100. Examples of power supplies include both internal and external power supplies. To meet the size and portability requirements, however, an internal power supply (i.e., a battery with a voltage ranging from about 1 to about 6 volts) can be used in the invention. If desired, more than a single power supply can be used.

Circuitry 112 contains all the necessary electrical components to convey the power from power supply 116 to light component 110. Depending on the number and types of light component(s) used and type of power supply, circuitry 112 can be adapted to provide the desired electrical pathway. In one aspect of the invention, circuitry 112 is kept as simple as possible and contains only a simple conducting line between the power supply 116 and light components 110. Of course, more complex circuitry could be used in the lighting means if needed.

Circuitry 112 is configured so that when access port 100 is not being used, light is not emitted. Because of size limitations, power supply 116 has a limited amount of power. To conserve that limited amount, circuitry 112 is configured so that light is only emitted when needed, i.e., when access port 100 is actively being used. Alternatively, lights 110 can be configured to blink when activated instead of being constantly provided power. The intermittent light pattern creates a high on-off contrast for enhanced visibility relative to a continuous light beam. In one aspect of the invention, this operation is performed by providing a circuitry 112 configured with additional components well known in the art to produce the pulsing light pattern when the circuit is closed. As with other circuit configurations previously described, when in the normal mode, the circuit is open so that no power flows from power supply 116 to the light components 110. When light is needed in an operational mode, circuit 112 is closed so that the power from power supply 116 flows to the light components 110.

In another modification, the port may be designed to emit visible light for a pre-determined time period following pressure activation by use of a timing circuit commonly known in the art. Closing the switch by applying pressure activates the timing circuit which transmits power to the LED for a specified period of time after which the timing circuit deactivates the switch, causing the LED to go off. The timing circuit may be programmed to maintain the switch in an activated state for a period of time sufficient to allow the practitioner to identify the septum and insert the needle, preferably between 5 and 20 seconds. A timing circuit provides an advantage over non-time activated designs in that it allows the practitioner to use both hands if desired to insert the needle since continual pressure is not required to maintain the circuit in a closed position.

There are numerous methods for configuring circuitry 112 to form an open circuit in a normal mode and to form a closed circuit in an operational mode. One example of such a method is depicted in FIGS. 13A-13B. In these Figures and as was previously described, circuitry 112 is incorporated into a means for separating its conductive elements from power supply 116. In a normal mode, the separating means keeps these components separate from each other. Separating means in these Figures comprises a flexible, insulating material with light components 110 mounted on (and supported by) an annular track 130 and interconnected by conductive pathway 132, wherein track 130 extends in a generally horizontal plane, and conductive pad/pressure switch 122 held in spaced, parallel relation to track 130 and bridged thereto by flexible arm 136. Separating means is then placed in access port 100 so that the end with light components 110 is in the desired emitting location (i.e., between the housing 102 and port can 106) and conductive pad/pressure switch 122 is near, but not contacting the power supply. In such a configuration, an open circuit is formed since the circuitry does not contact the power supply.

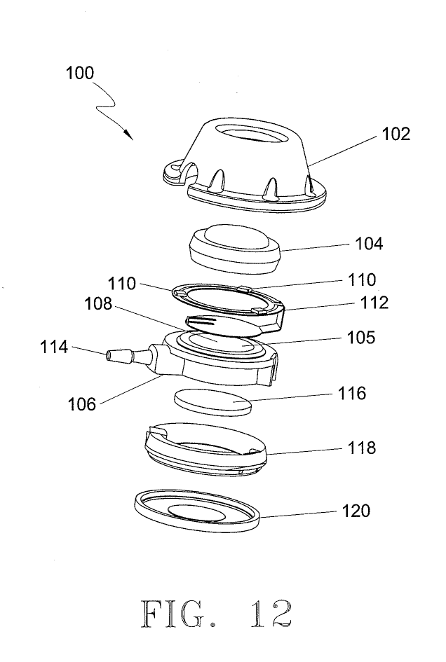

With such a configuration, to close the circuit and activate light components 110, a force is exerted against access port 100 (e.g., a force applied along vector P (FIG. 10) that is essentially perpendicular to the plane in which septum 104 extends). This action brings conductive pad/pressure switch 122 and power supply 116 into contact, closing the circuit and allowing power to flow through pathway 132 and hence, to light components 110, thereby emitting light. Once the force is removed, conductive pad/pressure switch 122 and power supply 116 are no longer in contact, the circuit is open, and with no power, light components 110 do not emit light.

The lighting means of the access port can be configured so that any type of force results in an emission of light. In one aspect of the invention, this force could be squeezing or pressing on the access port at any location. The amount of force needed to trigger the light emission can also vary from a slight tapping to a hard pressing.

In another aspect of the invention, the kinetic energy generated by the motion of normal body movement is stored in an internal holding cell such as a battery and implemented to provide a power source for the light emission. The patient's normal body movements are transformed into an electric current via a magnet and coil located within the port. The electrical current can then be stored using a capacitor or battery. Any other known means for storing and implementing the power generated from this kinetic energy can also be used.

In another aspect of the invention, the circuitry does not move as described above from an open position to a closed position. Instead, the light means is configured so that the application of an external electrical field (such as a capacitor or a wand) in effect closes the circuit and triggers the light emission. For example, a radiofrequency or microwave chip may be placed within the port which functions to activate a switch to close the circuit when an externally generated radiofrequency or microwave field is present (such as a field created by a RF or microwave wand). As well, the lighting means could be configured without a power supply and an external magnetic field could be applied to supply the necessary amount of power to actuate the light which would require use of a magnetic switch in the port. In these aspects where current is induced, obviously, batteries are not needed and the maintenance of the device is thereby enhanced. It should also be pointed out that external activation as described herein may also be used in combination with manual pressure activation to transmit power to the LEDs. In this aspect, the external activation provides a secondary means of activating the power which may be used in the event of a malfunction of the pressure activated switch or an inability to access the pressure points on the port due to port location deep within the tissue.

In one modification of the invention, structural components of the access port 100 (i.e., housing 102, septum 104, port can 106) can be made from any material that allows a greater amount of light to be emitted through it. Most materials used in access port components typically have a low degree of light transmittance. Examples of materials that can be used to improve light transmittance include translucent or transparent materials, such as glass, polyurethane, or polycarbonate. In the aspect of the invention where light components 110 are located between port can 106 and housing 102, the housing is made of such materials.

In another modification applicable to all embodiments, the light activating circuitry can be configured to turn the light emitting elements on and off at different time intervals or under different conditions as indicators. For example, the lighting means can be configured to indicate both the location of the septum and correct needle insertion into the septum. In this embodiment, the pressure-activated light component arrangement previously described may be used to indicate the precise location of the septum.

After locating the septum, the health practitioner inserts the needle through the patient's tissue and into the septum. Correct placement of the needle is indicated/verified by the illumination of an additional light emitting component, which is activated by the conductive coupling of a plate located on the bottom of the reservoir and conductive elements within the septum. The needle, which is conductive itself, acts as the switch to conductively couple the reservoir base plate and the conductive elements within the septum, thereby illuminating the light emitting component.

The septum may be made electrically conductive by the addition of a filler material such as silver, carbon or other conductive material commonly known in the art. Alternatively, a fine metallic mesh structure may be embedded within the septum body to act as the conductive element. In one embodiment, the bottom conductive plate may be eliminated by configuring a septum comprised of two horizontal planes of mesh (or other conductive filler) material. When the needle is inserted into the septum, it contacts both mesh planes thus completing the electrical circuit and allowing power to flow to the light emitting components.

The port of this invention (all embodiments) may also be configured to emit visible light when the port is impaired in some manner such as catheter occlusion or port leakage. A set of impedance-based sensors may be used to monitor and compare fluid-generated impedance within the port and externally in the tissue immediately surrounding the port. An impedance differential that is insignificant between the two locations may indicate that the port is leaking fluid to the surrounding tissue.

In another modification, the port may be configured with a pressure transducer located within the port, preferably on the bottom wall of the reservoir. The pressure transducer senses pressure levels and if a predetermined level is exceeded, the circuitry is automatically activated (the circuitry is also automatically actuated if the impedance-based sensors detect an impairment condition), causing the light emitting components to emit light as a visible alert of the impaired port. The predetermined pressure may be exceeded if for example, the catheter is partially or completely occluded or has become dislodged from the stem. The pressure transducer can also activate the circuitry in the event a medical practitioner attempts to inject fluid under high pressure into a port not designed for receiving high pressure injections. Alternatively or additionally, the impairment (impedance based or pressure transducer based) may be triggered when the medical personnel applies pressure to the port with a second light emitting element being actuated in the event an impairment is detected, with the second light emitting element having a distinct characteristic that differentiates it from the light emitting elements that define the position of the septum.

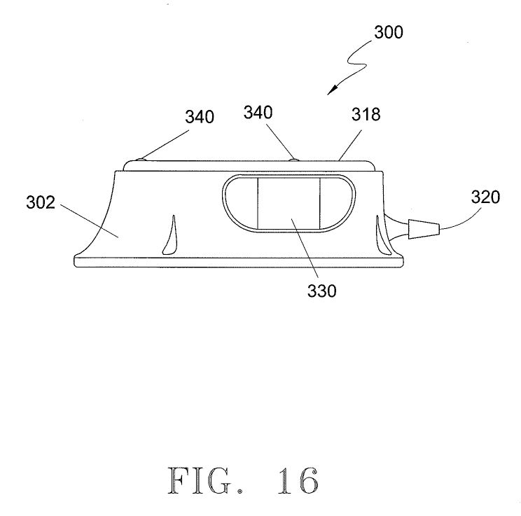

With reference to FIGS. 14-20, a vascular access port 300 constituting a third embodiment of the present invention is illustrated. Port 300 generally comprises an outer jacket 302 comprised of a flexible material, a main housing 304 situated within and including a body shape that contours outer jacket 302, a reservoir 306 formed in main housing 304, a pair of batteries 308, 310 that are securely positioned within main housing 304, an LED circuit 312 electrically coupled to batteries 308, 310, and that includes an opening 314 formed therethrough which is positioned concentrically around reservoir 306, a septum 316 that extends through opening 314 and in sealing relation to the open top of reservoir 306, and a cover 318 that is fixedly secured to main housing 304 and in covering relation to the other components of port 300. An exit lumen 320 extends outwardly from main housing 304, through outer jacket 302, and in fluid communication with reservoir 306.

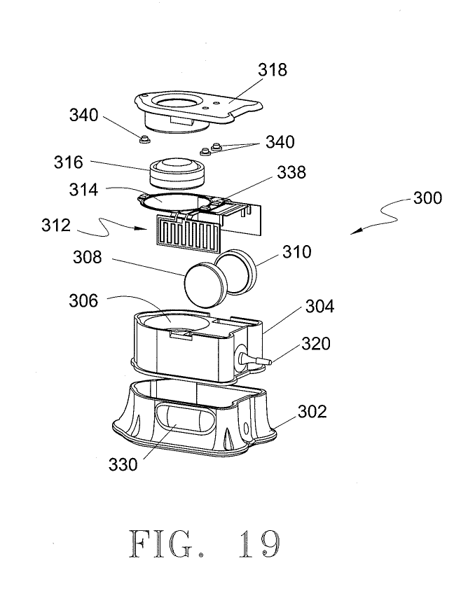

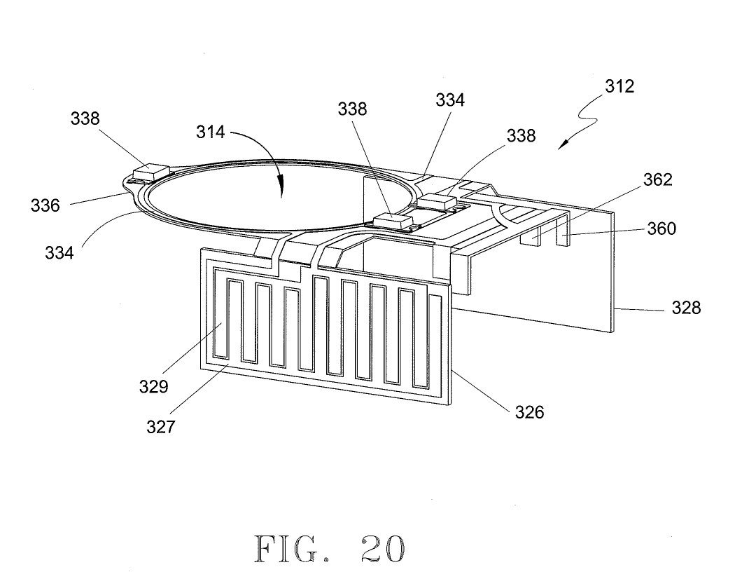

With reference to FIG. 18A and 18B, batteries 308, 310 are preferably of the disc-shaped type, although other types could be implemented as well, and are adapted to be securely positioned within vertical slots 322, 324, respectively, formed in main housing 304. Batteries 308, 310 are conductively connected to LED circuit 312 by positive and negative connections 360 and 362 respectively. LED circuit 312 includes a pair of panels 326, 328 that are positioned outwardly of adjacent batteries 308, 310, respectively, and inwardly adjacent user actuated buttons 330, 332, respectively, that are, in turn, positioned on opposing sides of outer jacket 302. The vertical position of the batteries is advantageous in that it minimized the overall height of the port, whereby increasing patient comfort.

Referring to FIG. 20, the light circuit 312 in its assembled form is shown. Light circuit 312 is comprised of panels 326, 328 include conductive serpentine traces 327, 329, respectively, etched on their outwardly facing surfaces that are electrically contiguous with the conductive pathway 334 formed on a light support track 336 that extends in bridging relation between conductive panels 326, 328, and in a plane transverse to the planes in which conductive panels 326, 328 extend. LEDs 338 are located on the upper surface of track 336 and may be of any acceptable type such as LEDs, incandescent, fluorescent, and the like.

When buttons 330, 332 are in their neutral (i.e., untouched) state, serpentine traces 327, 329 are not conductively coupled, thus maintaining an open circuit that will not transmit power through conductive pathway 334. The serpentine traces 327, 329 remain in a normally open state by use of either a spacer frame 337 or an air gap. As shown in FIG. 18B, spacer frame 337 is made of a non-conductive material such as plastic and is longitudinally positioned between the conductive plate 331 and the panel 326. The spacer frame is shaped like a picture frame with an outer rectangular perimeter of material that extends inwardly from the perimeter for approximately 0.040″ to form a solid border framing an open space. The spacer frame 337 is in contact with and supports the outer perimeter of the conductive plate 331, while preventing contact between the serpentine traces 327, 329 and the conductive plate 331.

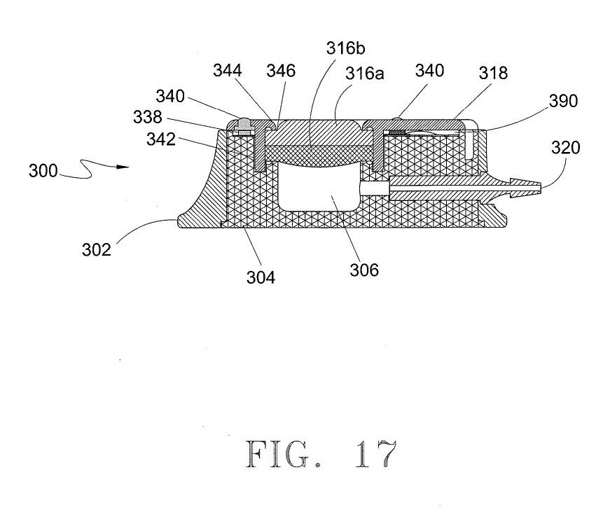

To activate the light circuit 312, buttons 330, 332 are manually depressed (e.g., by applying a force along vector T shown in FIG. 15 that is transverse to the longitudinal axis of port 300). Manual compression of the buttons causes the conductive plates 331, 333 to move toward and into conductive contact with the serpentine traces 327, 329 of panel 326, thus closing the circuit. Power is transmitted through the closed circuit created by conductive coupling of the serpentine traces 327, 329 on panels 326, 328 to conductive pathway 334, and ultimately to the plurality of light elements 338 that are positioned at spaced intervals along pathway 334.

Alternatively, the serpentine traces 327, 329 may be maintained in an open state by an air gap. Referring again to FIG. 18B, conductive plates 331, 332 may be bonded to the inner wall of lower housing 302 in a location vertically adjacent to the buttons 330, 332. Panels 326, 328 of the light circuit 312 are vertically positioned adjacent to the outer wall of main housing 304, as shown in FIG. 18A. A longitudinal open space or air gap between the panels 326, 328 and the conductive plates 331, 332 ensures that the circuit 312 remains in a normally open position. Pressure applied to buttons 330, 332 cause the inner surface of the conductive plates 331, 333 to move radially inward and into contact with the serpentine traces 327, 329 of panels 326, 328, thus closing the circuit and activating the light emitting elements.

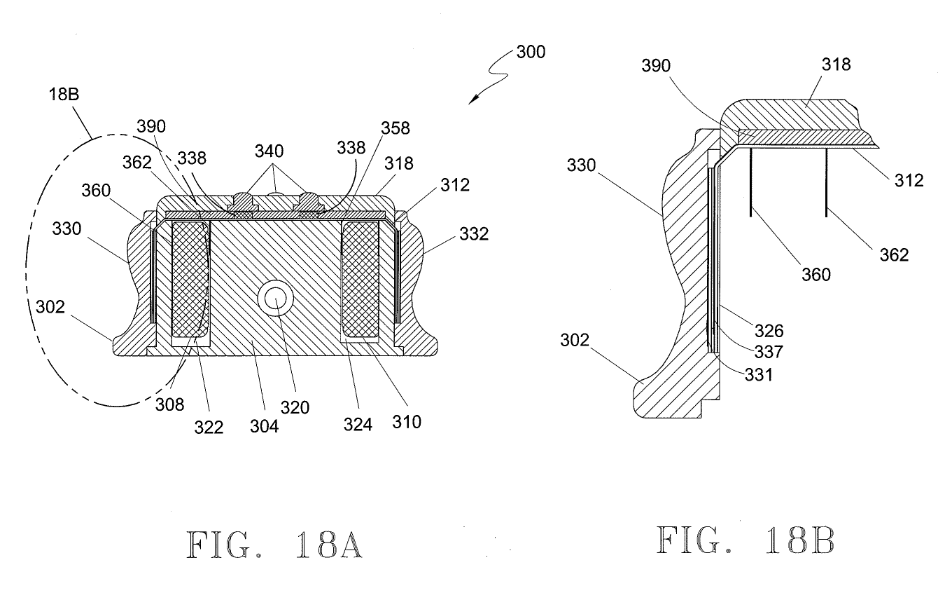

With reference to FIG. 17, where port 300 is shown in cross-section in its assembled condition, cover 318 is secured to main body 304 with light channeling elements (e.g., translucent members) 340 extending through openings formed therethrough and in covering relation to light emitting elements 338, thereby channeling the light emitted from light emitting elements 338 through the cover 318. Cover 318 further includes an annular extension 342 coming off its bottom surface that radially surrounds and supports septum 316, and a flange 344 that sits atop an annular shoulder 346 located adjacent to and in contact with the upwardly facing surface of septum 316. Extension 342 and flange 344 effectively secure septum 316 in a compressed and sealed position with main housing 304.

When assembled, port 300 includes space 390 which is defined by the upper surface of housing 304 and the lower surface of cover 318. Space 390 houses the LEDs 338 and portions of the circuitry 312. Space 390 may optionally be filled with an adhesive filler material such as epoxy to enhance the overall structural integrity of port 300, and specifically the sealing characteristics of the port. By filling space 390, the circuitry 312, LEDs 338 are held in sealing arrangement with the other port components, thus preventing moisture or fluid from entering space 390 and impairing port functionality.

Septum 316, as illustrated, may be composed of two portions of distinct material durometers 316a and 316b. If port 300 is to be used for injection of contrast media as is done for CT and other forms of imaging, material durometer 316a may be lower (i.e., softer) than durometer 316b. The use of a lower (softer) durometer 316b on the bottom layer when port 300 is used for injection of contrast media allows contrast media to be injected at relatively higher pressures than other forms of septum designs. The use of the lower (softer) durometer on the bottom layer of the septum will improve its efficacy in this regard. It is possible, however, that the reverse arrangement could be used, as could an arrangement where the harder material radially surrounds the softer material.

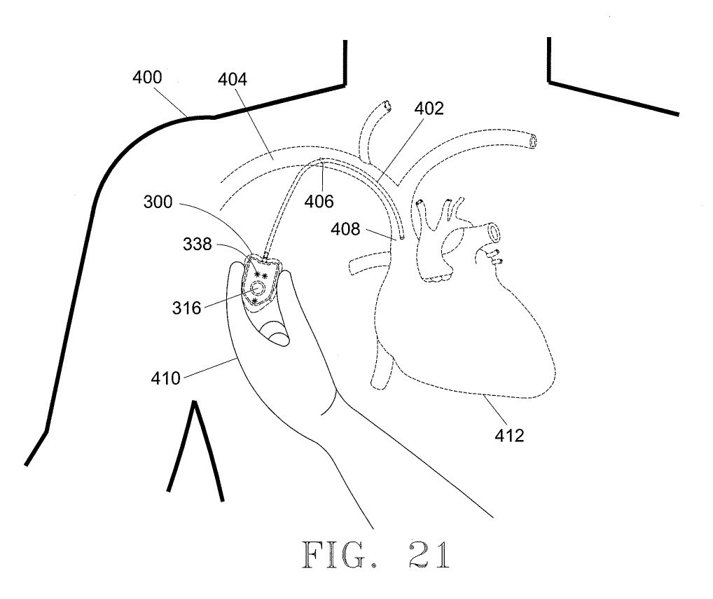

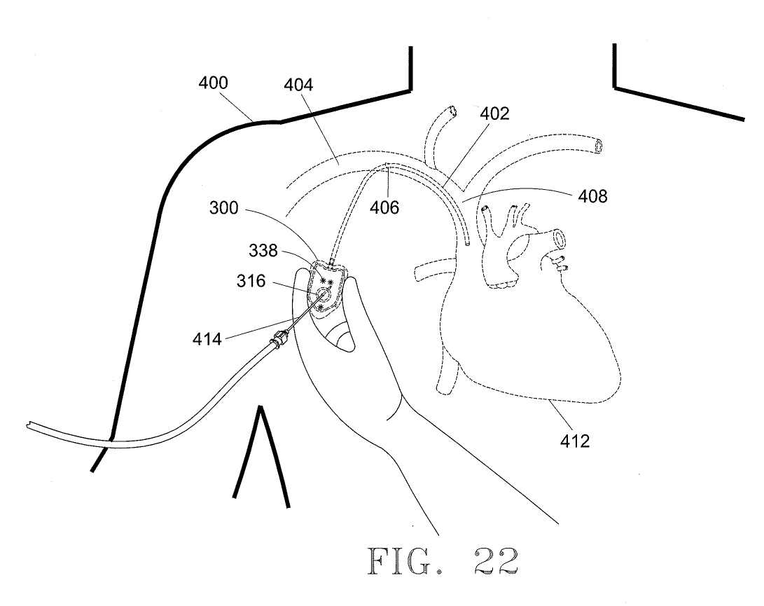

With reference to FIGS. 21-23, a method of non-invasively determining the location of the septum of an implanted vascular access port will be described. FIG. 21 illustrates a previously implanted vascular access port 300 within the subcutaneous chest tissue of patient 400, The port 300 includes a septum 316 and plurality of light emitting elements 338 which when activated are visible on the skin surface of patient 400. The port 300 is fluidly connected to catheter 402. Catheter 402 enters the subclavian vein 404 at entry point 406. The distal portion of the catheter is located at the junction of the superior vena cava 408 and the right atrium of the heart 412, where blood volume and flow rates are maximized.

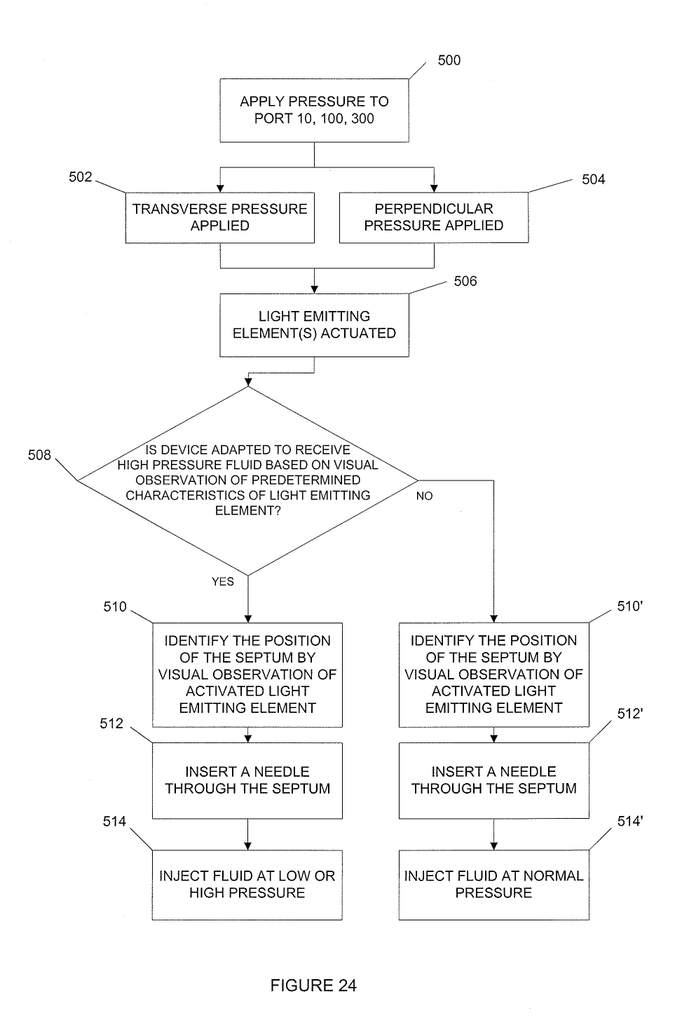

When injection of fluids or withdrawal of blood samples is needed, the medical practitioner activates the port light emitting elements 338 by applying manual finger pressure (step 500—see FIG. 24)) to the sides of the port (Step 502) as illustratively shown in FIG. 21 (or could compress the port (step 502′) if port 100 is being employed) and methodically shown in FIG. 24. Application of pressure to the port activates the circuit as previously described, causing the light emitting elements 338 to emit visible light (step 506) through the patient's tissue and skin surface. The practitioner may also use the light emitting elements to determine if the implanted port 300 is capable of withstanding increased pressures generated by high-pressure injection devices such as a CT injector (step 508). For example, the character of the activated light-emitting elements as described herein, may be used to indicate that the port includes a dual durometer or other type septum capable of withstanding high-pressure injection procedures. Conversely, the absence of light-emitting elements after the application of manual pressure would provide the health care professional with an indication that the port is not able to withstand higher injections.

Activation of the light emitting elements 338, which are located on the periphery of the septum 316, provides the medical practitioner with an immediate and accurate visual indicator of the septum location relative to the port 300 (step 510/510′). As illustrated in FIG. 22, the practitioner uses the light emitting elements to guide the insertion of the needle 414 tip into the port septum 316 (step 512/512′), and release the fluids into the port (step 514/514′). The presence of the lights on the periphery of the septum ensures that the practitioner will not mistakenly insert the needle outside of septum periphery. Thus, invention herein provides the practitioner with an easy, simple and instantaneous technique for non-invasively identifying the location of a septum and the type of implanted port without requiring additional activation or imaging equipment.

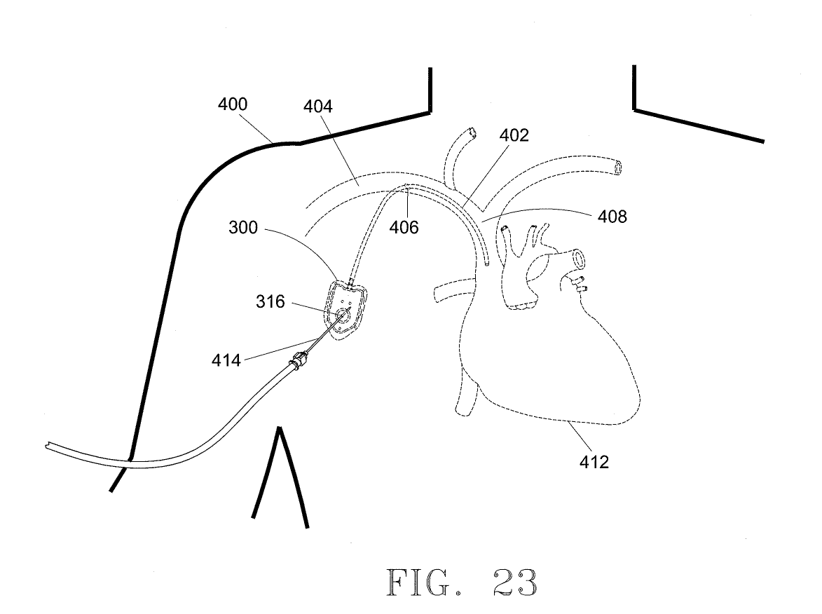

As illustrated in FIG. 23, after the practitioner has inserted the needle 414 through the skin into the septum 316, the light-emitting elements may be de-activated by removing manual finger pressure from the port. Alternatively, the circuitry actuating the light emitting elements may be configured such that the light emitting elements continue to emit light for a predetermined period of time following the release of pressure from the port, thereby permitting the visual identification to be enabled at a time when the medical personnel has both hands free to perform the procedure. Fluid injection or withdrawal can continue without the lights 338 being activated. If the needle becomes dislodged during injection or withdrawal, the medical practitioner may re-activate the light emitting elements to provide identification of the septum 316 location for re-insertion of needle 414.

In addition to any previously indicated variation, numerous other modification and alternative arrangements may be devised by those skilled in the art without departing from the spirit and scope of the invention and appended claims are intended to cover such modifications and arrangements. Thus, while the invention has been described above with particularity and detail in connection with what is presently deemed to be the most practical and preferred aspects of the invention, it will be apparent to those of ordinary skill in the art that numerous modifications, including but not limited to, form, function, manner of operations and use may be made without departing form the principles and concepts set forth herein.

What is claimed is:

1. A medical device suitable for subcutaneous implantation, comprising:
a. a housing;
b. a septum positioned in supported relation within said housing;
c. at least one light emitting element; and
d. light activating circuitry associated with said at least one light emitting element, wherein said circuitry is actuated by pressure applied to the medical device.

2. The medical device according to claim 1, further comprising a reservoir.

3. The medical device according to claim 2, further comprising a lumen extending outwardly from said reservoir.

4. The medical device according to clam 1, wherein said light activating circuitry comprises a light supporting member on which said at least one light emitting element is positioned.

5. The medical device according to claim 4, wherein said light supporting member is an at least partially annular track.

6. The medical device according to claim 1, wherein said septum is composed of at least first and second portions having first and second material durometers, respectively.

7. The medical device according to claim 6, wherein said first material durometer is lower than said second material durometer.

8. The medical device according to claim 7, wherein said first portion is radially surrounded by said second portion.

9. The medical device according to claim 7, wherein said first and second portions are layered relative to one another.

10. The medical device according to claim 9, wherein said first portion is positioned vertically above said second portion.

11. The medical device according to claim 9, wherein said first portion is positioned vertically below said second portion.

12. The medical device according to claim 1, further comprising at least one button associated with said housing and adapted for being depressed and activating said light activating circuitry when depressed.

13. The medical device according to claim 12, further comprising first and second buttons associated with said housing and adapted for being depressed.

14. The medical device according to claim 13, wherein said light activating circuitry is actuated upon depression of either of said first and second buttons.

15. The medical device according to claim 13, wherein said light activating circuitry is actuated upon depression of both of said first and second buttons.

16. The medical device according to claim 13, wherein said first and second buttons are positioned on opposing sides of said housing.

17. The medical device according to claim 1, wherein said light activating circuitry is selectively, manually actuable from an open position to a closed position by application of a transversely directed force.

18. The medical device according to claim 1, wherein said circuitry is selectively, manually actuable between an open position and a closed position by application of perpendicularly directed force.

19. The medical device according to claim 1, further comprising a switch operably associated with said light activating circuitry movable from an open position to a closed position.

20. The medical device according to claim 19, wherein said switch is movable between its open and closed positions by conductive coupling.

21. The medical device according to claim 20, wherein said conductive coupling comprises a first layer of conductive material associated with said septum and a second layer of conductive material in at least one of said septum and said housing, wherein said first and second layers of conductive material are electrically connected to said at least one light emitting element and are coupled by a conductive needle inserted through said septum.

22. The medical device according to claim 21, wherein said at least one light emitting element is adapted to emit light of a first color when actuated by said light activating circuitry and a second color when actuated by said switch.

23. The medical device according to claim 1, wherein said at least one light emitting element comprises at least a first light emitting element adapted to emit light of a first color, and a second light emitting element adapted to emit light of a second color.

24. The medical device according to claim 1, wherein said at least one light emitting element is adapted to blink upon being actuated.

25. The medical device according to claim 1, further comprising a power source for supplying power to said light activating circuitry.

26. The medical device according to claim 25, wherein said power source comprises a kinetic energy holding cell actuated by the body movement of the patient.

27. The medical device according to claim 25, wherein said power source is external to the patient and is adapted to induce current in said light activating circuitry.

28. The medical device according to claim 25, wherein said power source comprises at least one battery.

29. The medical device according to claim 28, wherein said at least one battery is positioned within said device to extend in a plane essentially parallel to that in which said septum extends.

30. The medical device according to claim 28, wherein said at least one battery is positioned within said device to extend in a plane that is transverse to that in which said septum extends.

31. The medical device according to claim 1, wherein said housing is composed of a material that permits light transmittance therethrough.

32. The medical device according to claim 31, wherein said at least one light emitting element is positioned relative to said housing such that it is adapted to emit light through said housing.

33. The medical device according to claim 1, wherein said light activating circuitry comprises:
a first portion that extends in a first plane and that includes a conductive pathway formed thereon;
a second portion that extends in a second plane parallel to said first plane; and
a third portion that extends between and interconnects said first portion and said second portion.

34. The medical device according to claim 33, wherein said light emitting elements are mounted on said first portion and in electrical communication with said conductive pathway.

35. The medical device according to claim 34, further comprising positive and negative terminals attached to said second portion.

36. The medical device according to claim 35, further comprising a power source adapted for placement between said first portion and said second portion and in operable association with said positive and negative terminals.

37. The medical device according to claim 1, wherein said light activating circuitry comprises:
a light supporting member that extends in a first plane and includes a conductive pathway formed thereon; and
a first panel extending in a first plane transverse and in connected relation to said first portion.

38. The medical device according to claim 37, wherein said at least one light emitting element is positioned on said light supporting member in electrical communication with said conductive pathway.

39. The medical device according to claim 37, further comprising a second panel connected to said light supporting member and extending in a third plane that is spaced from and parallel to said second plane.

40. The medical device according to claim 39, wherein said first and second panels each include respective conductive pathways formed thereon, each of which is electrically connected with said conductive pathway formed on said light supporting member.

41. The medical device according to claim 40, further comprising first and second power sources positioned in parallel relation adjacent to said first and second panels, respectively.

42. The medical device according to claim 41, further comprising a housing that includes first and second buttons formed thereon that are positioned outwardly adjacent said first and second panels, respectively.

43. The medical device according to claim 42, wherein said first and second buttons are movable by manually applied force thereto.

44. The medical device according to claim 1, wherein said light activating circuitry comprises:
a first portion that extends about said septum and includes a conductive pathway formed thereon; and
a second portion connected to said first portion and that includes positive and negative terminals mounted thereon.

45. The medical device according to claim 44, further comprising first and second pressure switches formed on said first portion.

46. The medical device according to claim 45, further comprising an upper housing positioned in surrounding relation to said housing, and with said first portion being positioned between said upper housing and said housing.

47. The medical device according to claim 46, wherein said upper housing includes first and second buttons formed thereon and positioned outwardly of and adjacent to said first and second pressure switches, respectively.

48. The medical device according to claim 1, wherein the medical device is a vascular access port.

49. The medical device according to claim 1, further comprising a second septum supported by said housing.

50. The medical device according to claim 1, wherein said light activating circuitry is adapted to illuminate said at least one light emitting element for a predetermined duration.

51. The medical device according to claim 1, wherein said light activating circuitry is adapted to illuminate said at least one light emitting element when the medical device becomes impaired.

52. The medical device according to claim 51, further comprising an impedance-based sensor associated with the medical device and adapted to monitor and compare fluid-generated impedance within the medical device and externally of the medical device in the tissue immediately surrounding the medical device.

53. The medical device according to claim 51, further comprising a pressure transducer located within the medical device and adapted to sense pressure levels within the medical device and, whereby if a predetermined pressure level is exceeded, said light activating circuitry is activated causing said at least one light emitting component to emit light.

54. The medical device according to claim 1, wherein said at least one light emitting element is an LED.

55. A medical device suitable for subcutaneous implantation, comprising:
a. a housing;
b. a septum positioned within and supported by said housing;
c. at least one light emitting element positioned in position defining relation to said septum;
d. light activating circuitry associated with said at least one light emitting element; and
e. a first switch operably associated with said light activating circuitry movable from an open position to a closed position by manually applied pressure.

56. A method for non-invasively determining the location of a medical device implanted subcutaneously in a patient, wherein the medical device comprises a housing, a septum positioned within and supported by said housing, at least one light emitting element, and light activating circuitry associated with said at least one light emitting element, the method comprising the step of applying pressure to the medical device, wherein the applied pressure actuates the at least one light emitting element.

57. The method according to claim 56, comprising the further step of identifying the position of the septum based upon visual identification of the position of the actuated light emitting element.

58. The method according to claim 57, comprising the further step of inserting a needle through said septum.

59. The method according to claim 56, comprising the further step of determining whether the medical device is adapted to receive an injection of high pressure fluid.

60. The method according to claim 56, wherein the step of applying pressure is done by applying force to the medical device in a direction that is transverse relative to the medical device.

61. The method according to claim 56, wherein the step of applying pressure is done by applying force to the medical device in a direction that is perpendicular relative to said septum.

62. The method according to claim 56, comprising the further step of determining whether the device is impaired.

63. The method according to claim 62, wherein the device includes a pressure transducer incorporated therein, and the step of determining whether the device has been impaired includes the step of actuating the at least one light emitting element in response to the pressure transducer determining that the pressure within the device exceeds a predetermined threshold.

64. The method according to claim 62, wherein the device includes an impedance-based sensor incorporated therein and the step of determining whether the device has been impaired includes the step of actuating the at least one light emitting element in response to an impedance differential between the interior of and exterior to the device exceeding a predetermined threshold.

65. The method according to claim 56, wherein the device includes a pressure transducer incorporated therein, comprising the further step of determining whether the device has received an injection of fluid at a pressure above a predetermined threshold and if it said threshold is exceeded, then actuating the light activating circuitry to cause the at least one light emitting element to emit light.

66. The method according to claim 56, comprising the further step of determining whether the device can safely receive high pressure fluid injections.

67. The method according to claim 66, wherein the step of determining whether the device can safely receive high pressure fluid injections includes including a second light emitting element that is actuable by the light activating circuitry and exhibits a distinct characteristic that distinguishes it from the at least one light emitting element.

68. A medical device suitable for subcutaneous implantation, comprising:
a. a housing;
b. a septum positioned within and supported by said housing and adapted to receive an injection of high pressure fluid therethrough;
c. a first light emitting element adapted for identifying the capacity of the medical device to receive high pressure fluid injections and emitting light of a first distinctive character;
d. a second light emitting element adapted for placement in position defining relation to said septum and emitting light of a second distinctive character; and
e. light activating circuitry associated with said first and second light emitting elements, wherein said circuitry is actuated by manually applied pressure.

69. The medical device according to claim 68, wherein said septum is composed of at least first and second portions having first and second durometers, respectively.

70. The medical device according to claim 69, wherein said first durometer is lower than said second durometer.

71. The medical device according to claim 70, wherein said first portion is radially surrounded by said second portion.

72. The medical device according to claim 70, wherein said first portion is layered on top of said second portion.

73. The medical device according to claim 70, wherein said first portion is layered below said second portion.

74. The medical device according to claim 68, wherein said housing is composed of a material that permits light to transmit therethrough.

75. The medical device according to claim 74, wherein said second light emitting element is positioned relative to said housing such that it is adapted to emit light therethrough.

76. The medical device according to claim 68, wherein said light activating circuitry is actuated by transversely directed force.

77. The medical device according to claim 68, wherein said light activating circuitry is actuated by force perpendicularly applied relative to said septum.

78. The medical device according to claim 68, wherein the medical device is a vascular access port.

79. The medical device according to claim 68, wherein said first distinctive character is a light of a first color.

80. The medical device according to claim 79, wherein said second distinctive character is a light of a second color.

81. A medical device suitable for a predetermined use and for subcutaneous implantation, comprising:
a. a housing;
b. a septum positioned within and supported by said housing, and having the capacity to be used for the predetermined use;
c. at least one first light emitting element associated with said housing and adapted to identify the capacity of the medical device to be used for the predetermined use; and
d. light activating circuitry.

82. The medical device according to claim 81, wherein said light activating circuitry is actuated by pressure.

83. The medical device according to claim 82, wherein said housing includes at least one button as a part thereof that upon being depressed actuates said light activating circuitry.

84. The medical device according to claim 83, wherein said light activating circuitry is actuated by transversely directed force applied to said at least one button.

85. The medical device according to claim 81, further comprising at least one second light emitting element associated with said housing and positioned in proximity to said septum to enable visual identification of said septum's location upon actuation of said at least one second light emitting element.

86. The medical device according to claim 82, wherein said septum is composed of at least first and second portions having first and second durometers, respectively.

87. The medical device according to claim 86, wherein said first durometer is lower than said second durometer.

88. The medical device according to claim 87, wherein said first portion is radially surrounded by said second portion.

89. The medical device according to claim 87, wherein said first portion is layered on top of said second portion.

90. The medical device according to claim 87, wherein said first portion is layered below said second portion.

91. A method for determining whether a medical device adapted for subcutaneous implantation in a patient has been impaired, wherein the medical device comprises a housing, a septum positioned within and supported by the housing, a reservoir positioned adjacent to the septum and adapted to receive a fluid therein, at least one light emitting element, and light activating circuitry operably coupled to the at least one light emitting element, the method comprising the steps of:
a. incorporating a predetermined sensor in the housing that is adapted to quantitatively measure a predetermined physical condition and compare said quantitative measurement to a predetermined threshold; and
b. actuating the light activating circuitry in the event said predetermined threshold has been exceeded, thereby causing the at least one light emitting element to emit light.

92. The method according to claim 91, wherein said predetermined sensor comprises a pressure transducer.

93. The method according to claim 91, wherein said predetermined sensor comprises an impedance-based sensor that compares the impedance of the fluid within the medical device to the impedance outside the medical device.
